(19) United States
(12) Patent Application Publication
(10) Pub. No.: US 2007/0073049 A1
(43) Pub. Date: Mar. 29, 2007

(54) METHOD AND APPARATUS FOR NUCLEIC PURIFICATION USING ION-PERMEABLE POLYMER-COATED ELECTRODE

(75) Inventors: Myo-Yong Lee, Suwon-si, Gyeonggi-do (KR); Jung Nam Lee, Namdong-gu, Incheon (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD., Suwon-si (KR)

(21) Appl. No.: 11/534,896

(22) Filed: Sep. 25, 2006

(30) Foreign Application Priority Data
Sep. 23, 2005 (KR) 10-2005-0088867

(51) Int. Cl. C07H 21/04

(52) U.S. Cl. 536/25.4; 204/450

(57) ABSTRACT

A method and apparatus for nucleic acid purification using an ion-permeable polymer-coated electrode are provided. The method for nucleic acid purification according to the present invention comprises the steps of exposing a fluid sample containing nucleic acids to an electrode coated with an ion-permeable polymer, applying a voltage to the electrode to attach the nucleic acids in the fluid sample thereto, removing the residual fluid sample from the electrode carrying the nucleic acids, and eluting the nucleic acids attached to the electrode into a buffer. The apparatus comprises an electrode coated with an ion-permeable polymer; and a voltage applying device for applying a voltage to the electrode.

This application claims priority to Korean Patent Application No. 10-2005-0088867, filed Sep. 23, 2005, and all the benefits accruing therefrom under 35 U.S.C. § 119, the contents of which in its entirety are herein incorporated by reference.

### BACKGROUND OF THE INVENTION

1. Field of the Invention

The invention relates to a method and apparatus for nucleic acid purification. hi particular, the invention relates to a method and apparatus for purification of a nucleic acid from a fluid sample, such as, for example, an analyte sample, a mixture containing cellular debris, or any other biomolecule mixture.

2. Description of the Related Art

Recently, as the importance of DNA analyzing techniques has become emphasized, the need for a more efficient technique for purifying nucleic acids from living organisms has also been regarded as important and such techniques have been developed with many modifications. In fact, techniques for efficiently purifying and concentrating DNA have proven to be useful in a variety of applications. For example, advances in recombinant DNA technology continually require the use of DNA in the form of probes, genomic DNA and plasmid DNA. Advances in diagnostics also utilize DNA in a variety of ways. For example, DNA probes are routinely used for detection and diagnosis of human pathogens, detection of genetic disorders and detection of food contaminants. DNA probes are also routinely used for locating, identifying and isolating DNAs of interest for genetic mapping, cloning and recombinant gene expression. Furthermore, efficient techniques for purifying and concentrating nucleic acids can be used to rapidly monitor and detect the presence of a pathogen infected in blood, which allows for more efficient medical treatments. Further, analyis of bacterial nucleic acid obtained from such a method can be useful in developing therapeutics or genetically engineered plants.

However, samples containing nucleic acids obtained from a living organism, such as blood or cell lysates, are generally complex and contain non-nucleic acid components. For example, the mixture can contain cell wall materials, proteins, polysaccharides and numerous other materials. To capture the nucleic acids contained therein has been a time consuming task which generally must be carried out before the nucleic acid can be used in other processes such as replication (or amplification) procedures or hybridization. Therefore, if the overall process for DNA purification from the isolation to the concentration can be conducted in a single vessel, i.e., on a single chip, it can be expected to significantly reduce the time and cost for such treatment.

There are numerous protocols for the purification and concentration of nucleic acid. One such purification method is disclosed in U.S. Pat. No. 5,342,931, which is generally directed to a method for purifying DNA by increasing hydroxyl groups on a silica structure by way of treating it with a strong alkaline solution, allowing DNA to bind to such treated silica surface under conditions of neutral pH, for example allowing the DNA to bind to such treated silica surface in TE, TAE or TBE buffer, and purifying DNA with hot water or buffer therefrom.

U.S. Pat. No. 5,693,785 teaches a method for purifying DNA using hydroxylated silica polymers. Generally, the method comprises increasing O groups on the silica surface by treating it with a strong alkaline solution, increasing hydroxyl groups thereon through acidification (pH 4-5), allowing DNA to bind to such treated silica surface under conditions of neutral pH, for example allowing the DNA to bind to such treated silica surface in TE, TAE or TBE buffer, and purifying DNA with hot water or buffer therefrom.

U.S. Pat. No. 5,707,799 discloses a detection device for determining the presence or the content of an analyte in a test sample, wherein the surface-treated structures are arranged on a plate to fix a reactant.

However, these prior art methods and apparatuses for DNA purification have a problem in that they indispensably require a process for chemically treating the surface of the plate before the binding of DNA.

Additionally, U.S. Pat. No. 6,794,130 discloses a method for purifying nucleic acids by way of the direct use of an electrode to overcome the aforementioned problems. The method purifies nucleic acids by exposing an electrode to a cell lysate, applying a certain voltage to the electrode to capture the nucleic acids directly thereon, and recovering the nucleic acids from the electrode by exposing the electrode carrying the captured nucleic acids directly thereon to a buffer. The method has relatively high efficiency for capturing the nucleic acids on the surface of an electrode however it shows very low elution efficiency, and thus, it has a drawback in that the purification efficiency of nucleic acids is not very high since the actual amount of nucleic acids recovered from the electrode is not large.

### SUMMARY OF THE INVENTION

In order to solve the aforesaid problems of the prior art, the present inventors have therefore endeavored to study a method for nucleic acid purification applicable to a chip, and have discovered that an electrode coated with an ion-permeable polymer shows strong binding affinity to nucleic acids when a voltage is applied thereto. Oxidative damage of the nucleic acids attached to the electrode coated with an ion-permeable polymer is avoided, and the nucleic acids can be very easily eluted fiom the electrode when no voltage is applied thereto, allowing for the purification of undamaged nucleic acids with a significantly high recovery efficiency.

Furthermore, the method and apparatus for nucleic acid purification provide the benefits of purifying nucleic acids from a fluid sample in an environment-friendly way without using any chaotropic salt or harmful organic solvent and of being easily applied to a lab-on-a-chip format.

In one embodiment, the invention is directed to a method for nucleic acid purification, comprising exposing a fluid sample containing nucleic acids to an electrode coated with an ion-permeable polymer; applying a voltage to the electrode to attach the nucleic acids to the electrode; removing the fluid sample from the electrode and attached nucleic acids; and eluting the attached nucleic acids from the electrode into a buffer by treating the electrode with the buffer.

In one embodiment, the invention is directed to an apparatus for nucleic acid purification using an ion-permeable polymer-coated electrode, comprising an electrode coated with an ion-permeable polymer; and a voltage applying device for applying a voltage to the electrode.

In another embodiment, the invention is directed to an apparatus for nucleic acid purification using an ion-permeable polymer-coated electrode, comprising a reaction chamber having an electrode coated with an ion-permeable polymer; and a voltage applying device for applying a voltage to the electrode.

### BRIEF DESCRIPTION OF THE DRAWINGS

FIG. 1 presents a schematic of an ion-permeable polymer used in the present invention at a molecular level;

FIG. 2 is a schematic diagram showing functional elements of an embodiment of an apparatus for nucleic acid purification according to the present invention; and

FIG. 3 is a graph showing the results of the DNA content measured in the Example according to the present invention compared with DNA content measured for the Comparative Example.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The present invention provides a method for nucleic acid purification using an ion-permeable polymer-coated electrode. The method comprises exposing a fluid sample containing nucleic acids to an electrode coated with an ion-permeable polymer, applying a voltage to the electrode to attach the nucleic acids to the electrode, removing the fluid sample from the electrode and attached nucleic acids, and eluting the nucleic acids attached to the electrode into a buffer after introducing the buffer into the electrode.

As used herein, the term “nucleic acid” includes deoxyribonucleic acid (DNA) and ribonucleic acid (RNA).

The method of the present invention employs an electrode coated with an ion-permeable polymer. The ion-permeable polymer used for the coating of the electrode is a copolymer containing both nonionic repeat units and ion containing repeat units. Preferably, the ion-penneable polymer is a polymer with a cation exchange property which shows high cation conductivity and resistance to strong acid or oxidizing agents.

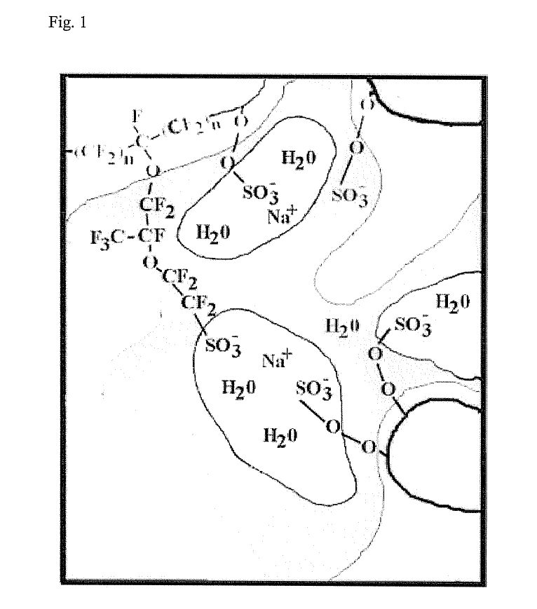

As illustrated in FIG. 1, such ion-permeable polymers are substances in which micro-phase separation occurs at a molecular level between a hydrophilic region containing the ion containing repeat units and the hydrophobic region containing the nonionic repeat units. Cation exchange transfer occurs in the hydrophilic region, which is called an ion cluster formed in a channel having a diameter of 1 nm, in which ionic groups in the ion containing repeat units are collected. In one embodiment, the ion containing repeat units include an ionic group such as —SO₃H or —COOH.

As illustrated in FIG. 1, since the ion-permeable polymer has the property that ions can permeate through the channel formed by the ion containing repeat units, the ion-permeable polymer itself does not show electrical conductivity, however it does allow ionic current to flow thereon due to the ion-conductivity.

There is no limitation to the type of ion-permeable polymer used if it has the aforesaid properties. In one embodiment, the ion-permeable polymer preferably comprises at least one material selected from the group consisting of a perfluorosulfonate ionomer with cation exchange capacity, a polysulfone, a polybenzimidazole and a polyetheretherketone.

The electrode coated with an ion-permeable polymer can be prepared by coating an electrode with the above-mentioned ion permeable polymer. Coating the electrode with an ion-permeable polymer can be accomplished by coating the ion-permeable polymer in the form of a resin directly onto a surface of the electrode or by attaching a film of the ion-permeable polymer to a surface of the electrode.

In order to purify nucleic acids from a fluid sample containing the same, the fluid sample containing the nucleic acids is first brought into contact with the ion-permeable polymer-coated electrode. Here, the fluid sample implies that nucleic acids are dispersed in a solvent such as a buffer. The fluid sample can include, for example, a cell lysate, an analyte sample, a mixture containing cellular debris, or any other biomolecule mixture. Other crude fluid samples from which to purify nucleic acids, especially DNA, include PCR or other amplification reaction mixes, sequencing reaction mixes, body fluid samples, e.g. blood or sputum or other DNA rich samples, e.g. micro-biological cultures.

When a certain positive voltage is applied to the ion-permeable polymer-coated electrode while the fluid sample containing nucleic acids is in contact with the electrode, the nucleic acids in the fluid sample migrate to the electrode to be attached thereto. In one embodiment, a positive voltage of up to about 2.5 V is applied to the ion-permeable polymer-coated electrode. If the applied voltage exceeds the above range, the nucleic acids attached to the electrode may oxidize and degrade, maling it impossible to purify undamaged nucleic acids. The positive voltage can be applied for any period suitable for maximizing attachment of the nucleic acids. In one embodiment, the time that the positive voltage is applied is 20 seconds to 10 minutes.

Following attachment of the nucleic acids to the ion-permeable polymer-coated electrode, the fluid sample is removed from the electrode. Through this step, components of the fluid sample that did not attach to the ion-permeable polymer-coated electrode are completely removed from the electrode without any chemical treatment.

After removing the fluid sample from the ion-permeable polymer-coated electrode according to the above step, the ion-permeable polymer-coated electrode carrying the attached nucleic acids is contacted with a buffer, thereby eluting the nucleic acids attached to the electrode into the buffer.

Elution of the attached nucleic acids from the electrode can be achieved in the presence or absence of an applied voltage field. When the electrode is exposed to the buffer, the nucleic acids attached to the ion-permeable polymer-coated electrode can elute into the buffer after a certain time period even if no voltage field is applied to the electrode. However, more efficient elution of the attached nucleic acids into the buffer can be achieved by applying a reversed voltage field to the electrode. This is because the nucleic acids attached to the ion-permeable polymer-coated electrode are more easily eluted into the buffer with high recovery efficiency when a negative voltage is applied to the ion permeable polymer-coated electrode carrying the nucleic acids.

The buffer used in the present invention may be any buffer that does not affect the nucleic acids' properties, including, for example, neutral pH buffers customarily used in nucleic acid reactions and manipulations. Examples include, TRIS/EDTA (TE) buffers, TRIS/acetate/EDTA (TAE) buffers, TRIS/borate (TB) buffers, TRIS/borate/EDTA (TBE) buffers, TRIS buffers, HEPES buffers, nucleic acid amplification buffers, and the like. In one embodiment, the buffer is a neutral pH buffer such as TE, TAE, or TBE. Instead of the buffer, water can be used.

The method of the invention may further comprise the step of washing the ion-permeable polymer-coated electrode carrying the nucleic acids with a wash buffer, between the step of removing the fluid sample and any of its components that fail to attach to the electrode and the step of eluting the attached nucleic acids into a buffer after introducing the buffer into the electrode. The additional step of washing the ion-permeable polymer-coated electrode carrying the nucleic acids enables more complete removal of unbound components of the fluid sample providing a purer nucleic acid product. The wash buffer can include, for example, TE buffers, TAE buffers, TB buffers, TBE buffers, TRIS buffers, HEPES buffers, nucleic acid amplification buffers, and the like.

Further, the invention provides an apparatus for nucleic acid purification using an ion-permeable polymer-coated electrode. In an embodiment, the apparatus comprises an ion-permeable polymer-coated electrode and a voltage applying device for applying a voltage to the electrode. In another embodiment, the apparatus comprises: a reaction chamber comprising an ion-permeable polymer-coated electrode; a voltage applying device for applying a voltage to the electrode; a sample storage chamber for storing a fluid sample comprising nucleic acids that is to be introduced into the reaction chamber; and a buffer storage chamber for storing a buffer that is to be introduced into the reaction chamber, e.g., after the introduced fluid sample is removed from the reaction chamber.

For the apparatus, the ion-permeable polymer-coated electrode is installed within the reaction chamber. Futhermore, it is preferable that the electrode is selected to maximize the total surface area of the ion-permeable polymer-coated electrode that can contact a fluid sample introduced into the reaction chamber. For example, an ion-permeable polymer-coated electrode having a microstnicture with the configuration of numerous pillars can be used.

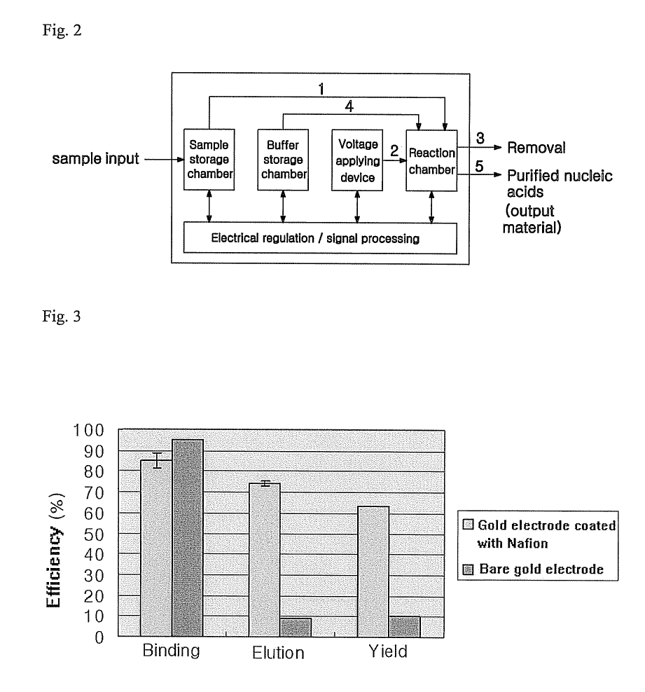

FIG. 2 illustrates functional elements of an embodiment of the apparatus for nucleic acid purification using an ion-permeable polymer-coated electrode. The embodiment shown in FIG. 2 includes four functional elements as follows: a reaction chamber having an ion-permeable polymer-coated electrode, a voltage applying device, a sample storage chamber, and a buffer storage chamber. Although not shown in FIG. 2, the inventive apparatus may further include microfluidic units connecting between the functional elements.

The apparatus for nucleic acid purification using an ion-permeable polymer-coated electrode may further include a purified material storage chamber for storing a buffer comprising nucleic acids eluted from the electrode of the reaction chamber

In one embodiment, the apparatus for nucleic acid purification of the present invention is used to purify nucleic acid from a fluid sample containing biomolecule. Therefore, the sample storage chamber is implemented in such a way that a fluid sample containing nucleic acids can be introduced from outside the apparatus and stored therein. The buffer storage chamber is also implemented in such a manner that a buffer can be introduced from outside the apparatus and stored therein.

In addition, the purified material storage chamber of the present invention may be configured to be connected to a device capable of performing nucleic acid amplification, e.g. a PCR chip, in order to permit eluted nucleic acids to be amplified by the device capable of performing nucleic acid amplification where necessary.

The microfluidic units disclosed herein include connecting parts that connect the sample storage chamber, the buffer storage chamber, and/or the purified material storage chamber to the reaction chamber. The microfluidic units can also include controlling parts which are formed between the reaction chamber and the sample storage chamber, the buffer storage chamber, and/or the purified material storage chamber that regulate the opening/closing operations of the connecting parts or each of the chambers in response to specific signals. The microfluidic units can also include an operating part for supplying a driving force for transferring fluids, e.g., a fluid sample, a wash buffer, or an elution buffer comprising the purified nucleic acids, between the above elements.

The microfluidic units used in the present invention are made using functional elements well-known in the art. In particular, it is preferable that the connecting part is a microchannel having a diameter sufficient to allow the nucleic acids within a fluid sample or a buffer to pass through. The controlling part is a flap valve, or a kind of active valve, which controls the flow rate of a fluid by opening/closing the flap by a driving force. The operating part can be a micropump.

With reference to the embodiment shown in FIG. 2, the role of each functional element in the apparatus and method for nucleic acid purification using an ion-permeable polymer-coated electrode will be described below in sequence. First of all, a sample containing nucleic acids is introduced into the reaction chamber and stored therein. A buffer is also introduced into the buffer storage chamber shown in FIG. 2 and stored therein. The sample introduced into the sample storage chamber is preferably in a fluid state, wherein the biomolecule containing nucleic acids is dispersed in a buffer. There is no limitation to the kind of fluid sample as long as it comprises the nucleic acids to be purified. An exemplary fluid sample includes a cell lysate. Further, in some embodiments, if the fluid sample contains whole cells, the fluid sample is subjected to cell lysis within the sample storage chamber, and the cell lysate obtained therefiom is introduced directly into the reaction chamber.

The fluid sample kept in the sample storage chamber is introduced into the reaction chamber through a connecting part at a certain amount. In some embodiments, the input amount of the fluid sample corresponds to an amount at which the reaction chamber is completely filled. The fluid sample is introduced at a constant flow rate. The flow rate chosen may differ depending on the surface area of the reaction chamber, but generally a flow rate is chosen such that the fluid sample is in contact with the electrode for about 20 seconds to about 10 minutes. Once the fluid sample has been introduced into the reaction chamber, a positive voltage of up to 2.5 V is applied from the voltage applying device to the ion-permeable polymer-coated electrode installed within the reaction chamber. Upon application of the voltage to the ion-permeable polymer-coated electrode the nucleic acids within the fluid sample move to the positive electrode and are attached to the surface thereof. The duration of time for applying a voltage to the ion-permeable polymer-coated electrode may differ depending on the content the fluid sample, or the amount of nucleic acid within the fluid sample, but may generally tale from about 20 seconds to about 10 minutes.

Subsequent to attachmnent of the nucleic acids to the ion-permeable polymer-coated electrode, the fluid sample is removed from the reaction chamber in order to remove components of the fluid sample which failed to attach to the electrode. Next, any residual fluid sample in the reaction chamber can be completely removed by performing an optional washing step. The washing step can be conducted by introducing a constant amount of the buffer kcept in the buffer storage chamber into the reaction chamber at a constant flow rate to fill the reaction chamber, followed by the immediate discharge of the buffer therefrom.

Elution of the attached nucleic acids can then be performed. In such an elution step, the constant amount of the buffer kept in the buffer storage chamber is introduced into the reaction chamber at a constant flow rate to fill the reaction chamber in order to elute the nucleic acids attached to the electrode into the buffer. If the reaction chamber is filled with the buffer and allowed to stand for a certain period of time (up to 10 minutes) without applying a voltage, the nucleic acids attached to the electrode spontaneously elute into the buffer, thereby resulting in the buffer containing the eluted nucleic acids. However, elution of the nucleic acids can be more rapidly and efficiently performed by applying a negative voltage to the electrode. The voltage can be applied for 1 seconds up to 10 seconds. In an embodiment, the negative voltage is identical in magnitude to the positive voltage initially applied to the electrode to induce attachment of the nucleic acids. When such a reverse voltage is applied to the electrode, the nucleic acids attached to the ion-permeable polymer-coated electrode are eluted into the buffer rapidly and with high elution efficiency.

In addition, although not shown in FIG. 2, the apparatus can further comprise a purified material storage chamber. Since the elution buffer, which is released from the reaction chamber and stored in the purified material storage chamber, contains only purified nucleic acids, the buffer comprising the purified nucleic acids can be transferred to another device connected thereto, such as a device capable of performing nucleic acid amplification, e.g. a PCR chip, for use as a reagent in subsequent experiments.

The apparatus for nucleic acid purification using an ion-permeable polymer-coated electrode has a structure such that it is feasible to implement each functional element in a lab-on-a-chip by employing well-known microfluidic techniques and MEMS devices.

The present invention will now be described in detail with reference to the following examples, which are not intended to limit the scope of the present invention.

### EXAMPLE

A DNA solution, which was prepared by dispersing E. coli genomic DNA (gDNA) in TE buffer at a concentration of 1 ng/μl, was employed as a fluid sample and introduced into a sample storage chamber. The fluid sample stored in the sample storage chamber was subjected to each step of the method for nucleic acid purification of the present invention in the apparatus for nucleic acid purification using an ion-permeable polymer-coated electrode of the present invention having the functional elements described in FIG. 2, and the buffer in which the nucleic acids were dispersed was obtained in the final step. Here, the ion-permeable polymer-coated electrode coated used in the apparatus for nucleic acid purification was prepared by coating a gold electrode with a 5% perfluorosulfonate ionomer (NAFION®) resin solution. The buffer introduced into the buffer storage chamber was TE buffer. In order to induce the attachment of DNA within the fluid sample to the electrode, a positive voltage of 2.5 V was applied thereto for 90 seconds. For the elution of the attached DNA from the electrode, a voltage of −2.5 V was applied to the electrode for 20 seconds.

During the above DNA purification procedure, the amount of DNA attached to the ion-permeable polymer-coated electrode, eluted therefrom and recovered in the buffer was measured for each step and normalized on the basis of the amount of DNA contained in the initial DNA solution. The results obtained are shown in FIG. 3.

### COMPARATIVE EXAMPLE

The DNA purification was canied out in the same condition as the aforesaid Example except that a bare gold electrode which was not coated with an ion-permeable polymer was employed. Similar to the above Example, the amounts of DNA were measured at each step (attached to the ion-permeable polymer-coated electrode, eluted therefrom, and recovered in the buffer) and normalized by the amount of DNA in the initial DNA solution. These results are also shown in FIG. 3.

Referring to FIG. 3, it was found that the efficiency of DNA attachment (“Binding”) to the ion-permeable polymer-coated electrode measured in the Example was similar to that for attachment to the bare gold electrode measured in the Comparative Example, but the efficiency of DNA elution into the buffer (“elution”) measured in the Example was significantly higher (about 7-fold or more) than that for elution into the buffer measured in the Comparative Example. The recovery efficiency (“yield”) of DNA was about 6-fold or more higher in the Example than in the Comparative example. Accordingly, it can be seen that the apparatus and method for nucleic acid purification using the ion-permeable polymer-coated electrode of the present invention has significantly higher purification efficiency of nucleic acids than an apparatus and method using a bare electrode having no coating.

As described above, the method and apparatus for nucleic acid purification using an ion-permeable polymer-coated electrode according to the present invention have several advantages as follows.

First, the method and apparatus for nucleic acid purification pennit purification of nucleic acids from a fluid sample by directly using an ion-permeable polymer-coated electrode.

Secondly, the method and apparatus for nucleic acid purification can purify nucleic acids from a fluid sample without the use of any chaotropic salts or harmful organic solvents.

Lastly, the method and apparatus for nucleic acid purification are easy to implement in a lab-on-a-chip format.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes and modifications may be made in the form and details of the described embodiments without departing from the spirit and scope of the invention as defined by the appended claims.

What is claimed is:

1. A method for nucleic acid purification, comprising:
exposing a fluid sample containing nucleic acids to an electrode coated with an ion-permeable polymer;
applying a voltage to the electrode to attach the nucleic acids to the electrode;
removing the fluid sample from the electrode and attached nucleic acids; and
eluting the attached nucleic acids from the electrode into a buffer by introducing the buffer into the electrode.

2. The method for nucleic acid purification according to claim 1, further comprising
applying a voltage to the electrode after introducing the buffer into the electrode,
wherein the voltage applied is a reverse of the voltage applied to the electrode to attach the nucleic acid.

3. The method for nucleic acid purification according to claim 1, further comprising
washing the electrode with a wash buffer after the fluid sample is removed from the electrode.

4. The method for nucleic acid purification according to claim 1, wherein the ion-permeable permeable polymer comprises a polymer with a cation exchange property.

5. The method for nucleic acid purification according to claim 4, wherein the ion-permeable polymer comprises an ionic group of —SO₃H or —COOH.

6. The method for nucleic acid purification according to claim 1, wherein the ion-permeable polymer comprises a perfluorosulfonate ionomer, a polysulfone, a polybenzimidazole, or a polyetheretherketone.

7. An apparatus for nucleic acid purification, comprising:
a reaction chamber comprising an electrode coated with an ion-permeable polymer; and
a voltage applying device for applying a voltage to the electrode.

8. The apparatus of claim 7, further comprising:
a sample storage chamber for storing a fluid sample comprising nucleic acids to be introduced into the reaction chamber; and
a buffer storage chamber for storing a buffer to be introduced into the reaction chamber.

9. The apparatus for nucleic acid purification according to claim 8, wherein the sample storage chamber comprises a means for performing cell lysis on a stored fluid sample.

10. The apparatus for nucleic acid purification according to claim 8, further comprising a purified material storage chamber for storing a buffer comprising nucleic acids purified from a fluid sample.

11. The apparatus for nucleic acid purification according to claim 10, wherein the purified material storage chamber is connected to a device capable of amplifying nucleic acid.

12. The apparatus for nucleic acid purification according to claim 7, wherein the ion-permeable polymer comprises a polymer with cation exchange property.

13. The apparatus for nucleic acid purification according to claim 12, wherein the ion-permeable polymer comprises an ionic group of —SO₃H or —COOH.

14. The apparatus of claim 7, wherein the ion-permeable polymer comprises a perfluorosulfonate ionomer, a polysulfone, a polybenzimidazole, or a polyetheretherketone.

15. The apparatus of claim 14, wherein the ion-permeable polymer comprises a perfluorosulfonate ionomer.

16. The apparatus of claim 7, wherein the electrode is a gold electrode.

17. The apparatus of claim 7, wherein the electrode is coated with the ion-permeable polymer by coating a resin of the ion-penneable polymer on a surface of the electrode.

18. A lab-on-a-chip comprising the apparatus for nucleic acid purification
